(19) United States
(12) Patent Application Publication
(10) Pub. No.: US 2022/0062921 A1
(43) Pub. Date: Mar. 3, 2022

(54) METHOD OF PREVENTING DAMAGE OCCURRING IN MICROBIAL AEROSOLIZATION PROCESS

(71) Applicant: Industry-Academic Cooperation Foundation, Yonsei University, Seoul (KR)

(72) Inventors: Jung Ho HWANG, Seoul (KR); Hyeong Rae KIM, Seoul (KR); Sang Mo KANG, Seongnam-si (KR)

(21) Appl. No.: 17/461,958

(22) Filed: Aug. 30, 2021

(30) Foreign Application Priority Data
Aug. 31, 2020 (KR) 10-2020-0110030

(51) Int. Cl. B05B 1/02

(52) U.S. Cl. CPC B05B 1/02

(57) ABSTRACT

A method of preventing damage generated in a microbial aerosolization process is provided. The method includes a first step of injecting microorganisms into an atomizer containing a phosphate-buffered saline (PBS) solution in which one or more selected from the group consisting of ascorbic acid (AA) and bovine serum albumin (BSA) are contained; and a second step of injecting compressed air into an atomizer and spraying a bioaerosol.

### CROSS-REFERENCE TO RELATED APPLICATION

This application claims priority under 35 U.S.C. § 119(b) to Korean Patent Application No. 10-2020-0110030, filed on Aug. 31, 2020, the disclosure of which is incorporated herein by reference in its entirety.

### BACKGROUND OF THE INVENTION

### 1. Field of the Invention

The present invention relates to a method of preventing damage generated in a microbial aerosolization process.

### 2. Discussion of Related Art

In the atmosphere, there are various fine materials other than air, and particles of biological origin such as molds, bacteria and pollen, i.e., airborne microorganisms (bioaerosols or bio-fine dust) are also included [Non-Patent Document 0001, 0002]. Bioaerosols present in the atmosphere are released from various sources in indoor and outdoor environments.

Bioaerosols are pointed out as a cause of air pollution and diseases, and in the human body, act as a cause of infectious diseases, allergies and respiratory diseases and the like [Non-Patent Document 0003˜0005]. Therefore, there is a need for technology of quickly and effectively detecting harmful bioaerosols [Non-Patent Document 0006].

Methods of detecting a bioaerosol include a method of measuring the number of colonies by sampling a bioaerosol in the air, spreading and culturing the bioaerosol and a method of measuring the autofluorescence of microorganisms [Non-Patent Document 0007, 0008].

To study such a bioaerosol, a method of dispersing a bioaerosol in a certain space with a desired material and a desired composition is constantly being attempted [Non-Patent Document 0009]. As a widely used method to simulate airborne microorganisms (bioaerosol or bio-fine dust), a method of aerosolizing microorganisms in the air using an atomizer is used. However, during the aerosolization process, approximately 90% of microorganisms are damaged due to inertial collisions and oxidation and lose vitality, and thus there is a limit to simulating microorganisms living in the air [Non-Patent Document 0009].

### RELATED ART DOCUMENTS

### Non-Patent Document

(Non-Patent Document 0001) Environ. Int. 2015, 85, 254-272.

(Non-Patent Document 0002) Aerosol Sci. Technol. 2017, 51 (7), 787-800.

(Non-Patent Document 0003) Analyst 2011, 136 (22), 4641-4652.

(Non-Patent Document 0004) J. Aerosol Sci. 2018, 115, 108-112.

(Non-Patent Document 0005) Int J Hyg Environ Health, 2015 October; 218(7): 577-89.

(Non-Patent Document 0006) Kim, H. R., An, S., & Hwang, J. Aerosol-to-hydrosol sampling and simultaneous enrichment of airborne bacteria for rapid biosensing. ACS Sensors. (2020).

(Non-Patent Document 0007) Journal of Hazardous Materials, Vol. 369, 684-690, 2019.

(Non-Patent Document 0008) Journal of Aerosol Science, Vol. 115, 190-197, 2018.

(Non-Patent Document 0009) Journal of Food Engineering, 113(2), 194-200, 2012.

### BRIEF SUMMARY OF THE INVENTION

The present invention is directed to providing a method of preventing damage generated in a microbial aerosolization process.

To achieve the above-described purpose, the present invention provides a microbial aerosolization method, which includes: a first step of injecting microorganisms into an atomizer containing a phosphate-buffered saline (PBS) solution in which one or more selected from the group consisting of ascorbic acid (AA) and bovine serum albumin (BSA) are contained; and a second step of injecting compressed air into an atomizer and spraying a bioaerosol.

In the present invention, the microorganisms may be bacteria, fungi or viruses.

In the present invention, the concentration of ascorbic acid (AA) may be 1 to 2.5 mg/mL.

In the present invention, the concentration of bovine serum albumin (BSA) may be 0.05 to 0.15 mg/mL.

In the present invention, the injection flow rate of compressed air may be 6 to 10 LPM.

### BRIEF DESCRIPTION OF THE DRAWINGS

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

The patent or application file contains at least one drawing executed in color. Copies of this patent or patent application publication with color drawings will be provided by the Office upon request and payment of the necessary fee.

FIG. 1 is a schematic diagram of an entire process;

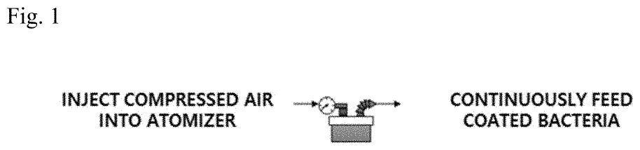

FIG. 2 is a microbial viability graph after aerosolization with 1.76 mg/ml ascorbic acid (AA) and 0.1 mg/ml bovine serum albumin (BSA); and

FIG. 3 is a microbial viability graph after aerosolization with 2.5 mg/ml ascorbic acid (AA) and 0.05 mg/ml bovine serum albumin (BSA).

### DETAILED DESCRIPTION OF THE INVENTION

Hereinafter, the present invention will be described in detail.

The present invention relates to a method of preventing damage generated in a microbial aerosolization process.

The method of preventing damage generated in a microbial aerosolization process according to the present invention may include: a first step of injecting microorganisms into an atomizer containing a phosphate-buffered saline (PBS) solution in which one or more selected from the group consisting of ascorbic acid (AA) and bovine serum albumin (BSA) are contained; and a second step of injecting compressed air into an atomizer and spraying bioaerosol.

The “atomizer” used herein refers to a spray nozzle used when humidifying or cleaning with a device that discharges water or steam into the air.

The “bioaerosol” used herein includes all gaseous materials derived from gaseous microorganisms or living organisms. For example, bioaerosols include living or dead microorganisms (bacteria or viruses), microbial debris, mold spores, pollen, allergens from plants and animals, coughs and body fluids from the human body, and toxins generated from microorganisms. Bioaerosols are infinitely abundant in nature, and are present in various places such as inside and outside houses, inside and outside buildings, and habitats of animals and plants. In addition, the sizes of the bioaerosols range from less than 1 micron to 100 microns.

First, microorganisms are injected into an atomizer in which there is a phosphate buffered saline (PBS) solution containing one or more selected from the group consisting of ascorbic acid (AA) and bovine serum albumin (BSA).

The microorganisms may be, but are not limited to, bacteria, fungi or viruses.

Phosphate buffered saline (PBS) is a commercially available product (lx, PBS buffer, Biosesang). Components of the corresponding solution are as follows: 137 mM sodium chloride, 2.7 mM potassium chloride, 4.3 mM sodium phosphate (dibasic, anhydrous), 1.4 mM potassium phosphate (monobasic, anhydrous), sterile solution.

The concentration of ascorbic acid may be 1 to 2.5 mg/mL, preferably, 1.5 to 2 mg/mL, and more preferably 1.76 mg/mL. The unit of the corresponding concentration is (mg ascorbic acid)/(ml PBS solution).

The concentration of bovine serum albumin (BSA) may be 0.05 to 0.15 mg/ml, preferably 0.75 to 1.25 mg/ml, and more preferably 0.1 mg/ml. The unit of the corresponding concentration is (mg bovine serum albumin (BSA))/(ml PBS solution).

Afterward, the bioaerosol is sprayed by injecting the compressed air into the atomizer containing a microorganism-injected PBS solution.

The injection flow rate of the compressed air may be 6 to 10 LPM, preferably 6 to 8 LPM, and more preferably 6 LPM.

The compressed air is not aerosolized at less than 6 LPM, and the survival rate of microorganisms is drastically lowered at 10 LPM or more.

Hereinafter, to help in understanding the present invention, preferable examples and experimental examples are provided. However, the following examples and experimental examples re merely provided to more easily understand the present invention, and not to limit the present invention.

### <Example 1> Bacterial Survival Rate after Bioaerosolization According to Air Injection Flow Rate

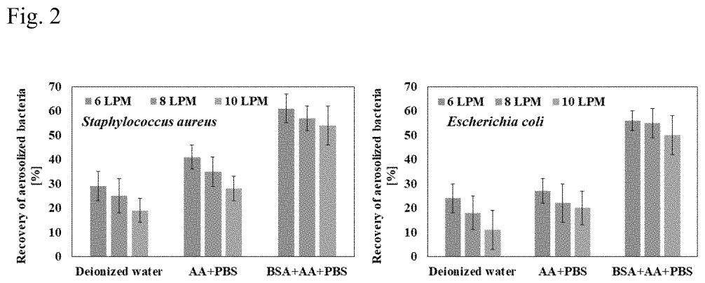

To aerosolize a prepared bacterial solution, the solution was injected into a water container of an atomizer (manufactured in-laboratory). Here, the water container of the atomizer contains 1.76 mg/ml ascorbic acid (AA) and 0.1 mg/ml bovine serum albumin (BSA) (mg AA or BSA/ml PBS solution) along with a phosphate-buffered saline (PBS) solution. When compressed air was injected into the atomizer over a certain flow rate, bacteria and a BSA solution met around an orifice so that the bacteria were coated with BSA and continuously fed with the atomizer. The experimental results are shown in FIG. 2.

Here, the survival rate of bacteria was higher than when bacteria were sprayed using conventional deionized water. Since ascorbic acid (AA) prevents the oxygen component of compressed air from oxidizing and killing bacteria, it causes the survival rate of the bacteria to slightly increase. Since bovine serum albumin (BSA) prevents dehydration in an aerosolization process by coating bacteria, it causes the survival rate of bacteria to increase. As the flow rate of compressed air increased, bacteria collided with a collision plate used for aerosolization inside the atomizer, and mechanical damage was also increased, resulting in reduction of the survival rate. Since the cell wall composition of gram-positive bacteria is stronger to mechanical and chemical stress than that of gram-negative bacteria, the gram-positive bacteria showed a higher survival rate.

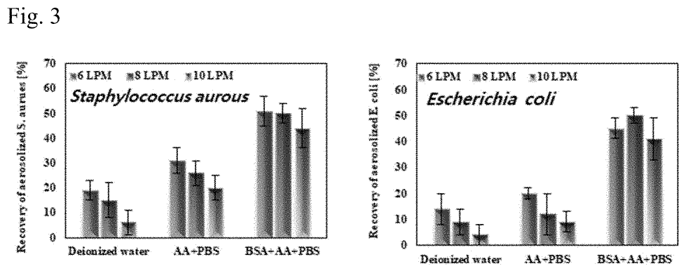

Subsequently, the results obtained when 2.5 mg/ml ascorbic acid (AA) and 0.05 mg/ml bovine serum albumin (BSA) are contained in a PBS solution are shown in FIG. 3.

In the above results, the pattern of the survival rate was generally the same as that of the results of FIG. 2, but the survival rate was low. Therefore, to increase the survival rate in the PBS solution, it is important to add appropriate amounts of AA and BSA.

According to the present invention, a method of preventing damage generated in a microbial aerosolization process can be provided.

In addition, the method of the present invention greatly reduces the damage rate of microorganisms due to inertial collisions or oxidation in an aerosolization process.

It will be appreciated by those skilled in the art that changes could be made to the embodiment described above without departing from the broad inventive concept thereof. It is understood, therefore, that this invention is not limited to the particular embodiments disclosed, but it is intended to cover modifications within the spirit and scope of the present invention as defined by the appended claims.

1. A microbial aerosolization method, comprising:
a first step of injecting microorganisms into an atomizer containing a phosphate-buffered saline (PBS) solution in which one or more selected from the group consisting of ascorbic acid (AA) and bovine serum albumin (BSA) are contained; and
a second step of injecting compressed air into an atomizer and spraying a bioaerosol.

2. The method of claim 1, wherein the microorganisms are bacteria, fungi or viruses.

3. The method of claim 1, wherein, in the first step, the concentration of the ascorbic acid is 1 to 2.5 mg/ml.

4. The method of claim 1, wherein, in the first step, the concentration of the bovine serum albumin (BSA) is 0.05 to 0.15 mg/ml.

5. The method of claim 1, wherein, in the second step, the injection flow rate of the compressed air is 6 to 10 LPM.
